(19) United States
(12) Patent Application Publication
(10) Pub. No.: US 2015/0164867 A1
(43) Pub. Date: Jun. 18, 2015

(54) METHODS OF IMPROVING LONG-TERM SURVIVAL AND REDUCING HOSPITALIZATION READMISSION RATES FOR SUBJECTS SUFFERING FROM HEPATIC ENCEPHALOPATHY

(71) Applicant: Salix Pharmaceuticals, Ltd, Raleigh, NC (US)

(72) Inventor: Guy Neff, Tampa, FL (US)

(21) Appl. No.: 14/629,871

(22) Filed: Feb. 24, 2015

Related U.S. Application Data
(63) Continuation of application No. PCT/US2013/059590, filed on Sep. 13, 2013.
(60) Provisional application No. 61/700,862, filed on Sep. 13, 2012.

(51) Int. Cl. A61K 31/437

(52) U.S. Cl. CPC A61K 31/437

(57) ABSTRACT

Methods of increasing the long-term survival odds for a subject suffering from hepatic encephalopathy are provided, wherein the methods include administering a composition comprising rifaximin to the subject. Methods of reducing the frequency of hospitalization visits by a subject suffering from HE are also provided, wherein the methods include administering a composition comprising rifaximin to the subject.

### RELATED APPLICATIONS

This application claims the benefit of International Application No. PCT/US2013/059590, filed on Sep. 12, 2013, which claims benefit of U.S. Provisional Application No. 61/700,862, filed on Sep. 13, 2012. The entire contents of each of the aforementioned applications are incorporated herein by reference.

### BACKGROUND

Hepatic encephalopathy (HE) is caused by a reversible decrease in neurologic function associated with liver failure and portosystemic venous shunting. HE occurs in 1 of every 3 cases of cirrhosis, in cases of fulminant hepatic failure reported in the United States (US), and is present in nearly half of patients reaching end-stage liver disease. It may occur at any age, but the peaks parallel those of fulminant liver disease (peak=40's), and cirrhosis (peak=late 50's).

The incidence of HE is likely to increase with the incidence of hepatitis C in the general population and cirrhotics in aging patients. Acute HE signifies a serious prognosis with a 40% likelihood of survival for 1 year. Fatigue is also a very common complaint in patients suffering from all stages of cirrhosis. One common cause that leads to fatigue in this patient population is sarcopenia (muscle loss). Several factors can lead to sarcopenia, including decreased caloric intake culminating in malnutrition.

Accordingly, there is a need in the art for a compositions and methods for treating and preventing HE, including increasing the long-term survival of patients suffering from HE. There is also a need to improve the quality of life in patients suffering from HE, such as, for example, reducing the frequency of hospitalization visits and addressing fatigue and sarcopenia in such patients.

### SUMMARY

Embodiments are directed to a method of improving the survival outcome for a subject suffering from hepatic encephalopathy (HE), wherein the methods include administering a composition comprising rifaximin to the subject. In some embodiments, the survival outcome is improved by at least about 5%, 10%, 25%, 50% or 75% relative to a subject that has not been treated with rifaximin.

Embodiments also relate to a method of reducing the frequency of hospitalization visits by a subject suffering from HE, wherein the methods include administering a composition comprising rifaximin to the subject. In some embodiments, the reduction in the frequency of hospitalization visits comprises a reduction in the frequency of hospital readmissions.

Embodiments also relate to a method of decreasing the risk of developing sarcopenia in a subject suffering from cirrhosis and hepatic encephalopathy, wherein the method includes administering a composition comprising rifaximin to the subject.

Embodiments are also directed to a method of maintaining normal daily caloric intake in a subject suffering from cirrhosis and hepatic encephalopathy, wherein the method includes administering a composition comprising rifaximin to the subject. In some embodiments, the subject maintains at least about 75%, 80%, 85% or 90% of normal daily caloric intake. In some embodiments, administration of rifaximin reduces the risk of developing sarcopenia in the subject. In some embodiments, administration of rifaximin reduces the risk of suffering from malnutrition in the subject.

Embodiments are directed to a method of decreasing the risk of suffering from malnutrition in a subject suffering from cirrhosis and hepatic encephalopathy, wherein the method includes administering a composition comprising rifaximin to the subject.

In some embodiments, the subject is not concurrently administered lactulose.

In some embodiments, the subject is administered rifaximin at a dose of about 50 mg to about 6000 mg per day.

In some embodiments, the subject is administered rifaximin at a dose of between about 100 mg and about 6000 mg; from between about 50 mg and about 2500 mg BID; from between about 50 mg and about 2000 mg TID; 200 mg TID; 200 mg BID or 200 mg QD.

In some embodiments, the subject is administered rifaximin at a dose of about 550 mg, 600 mg or 1650 mg TID, QD or BID.

In some embodiments, the subject is administered rifaximin at a dose of about 550 mg BID.

In some embodiments, the subject is administered the composition for between about 1 week and about 24 months.

In some embodiments, the subject is administered the composition for about 14 days.

In some embodiments, the subject is administered the composition for at least about six months.

In some embodiments, the subject is administered the composition for at least about twelve months.

In some embodiments, the subject is administered the composition for at least about 24 months.

In some embodiments, the subject is administered the composition for at least about 36 months.

In some embodiments, the subject is administered the composition for the duration of the subject's life.

In some embodiments, the composition does not comprise lactulose.

### DETAILED DESCRIPTION

Hepatic encephalopathy, also known as hepatic coma or portal-systemic encephalopathy (PSE), is a serious, rare, complex, episodic, neuropsychiatric syndrome associated with advanced liver disease. Hepatic encephalopathy is a formidable burden on the patient, his/her family, and the healthcare system; and the current standard of care is inadequate. Overt, episodic HE is common among patients with liver cirrhosis. The condition is rare among individuals in the overall, general population. Overt HE episodes are debilitating, can present without warning, render the patient incapable of self-care, and frequently result in hospitalization. The frequency of hospitalizations due to HE increased since 1993 to over 40,000 patients in 2003; and in 2004, 50,962 patients were hospitalized with a principal diagnosis of HE. HE, as used herein, comprises, for example, episodic, persistent and minimal HE.

Fatigue is also a very common complaint in patients suffering from all stages of cirrhosis, including those suffering from subclinical hepatic encephalopathy (SHE). One common cause that leads to fatigue in this patient population is sarcopenia (muscle loss). Several factors lead to sarcopenia, including decreased caloric intake culminating in malnutrition. It is known that compensated cirrhotics consume only nearly 80% of their daily-recommended calories. Thus the loss in daily calories in cirrhotic patients magnifies the decrease in quality of life and enhances sarcopenia and deconditioning, thus intensifying fatigue. Disclosed herein is a study that includes evaluation of the daily caloric intake of compensated cirrhotic patients undergoing subclinical hepatic encephalopathy (SHE) management.

The main pathogenesis of HE is related to nitrogenous substances derived from the gut adversely affecting brain function. The most influential of these compounds is thought to be ammonia, a byproduct of protein digestion that is normally detoxified by the liver. Correlation of blood levels with mental state in cirrhosis, however, is inaccurate, in part, because the blood-brain barrier permeability to ammonia is increased in patients with HE. Other gut-derived toxins have also been proposed as being responsible for HE.

In patients with chronic liver disease, the occurrence of hepatic encephalopathy is associated with a low quality of life compared to age-matched patients without HE. Overt HE episodes are debilitating, can present without warning, render the patient incapable of self-care, and frequently result in hospitalization. Patients with HE experience symptoms including fatigue, daytime sleepiness, and lack of awareness (Conn score 1); and confusion and disorientation (Conn score 2) that significantly interfere with day-to-day function and decreased ability for self care. Often, this lack of self care leads to improper nutrition and non-adherence to therapy and further escalates into more severe symptoms such as increased somnolence, gross disorientation and stupor (Conn score 3) or coma (Conn score 4).

A history of overt HE episodes and the severity of HE episodes were also found to be predictive of decreased survival in patients with chronic liver disease. In patients with liver cirrhosis and a history of overt HE episodes, survival probability was 42% at 1 year and 23% at 3 years after experiencing an HE episode. In another analysis, the occurrence of an HE episode of Conn score 2 in patients with cirrhosis was associated with a 4-fold increase in the risk of death.

It has been surprisingly discovered that, among the various HE therapies, administration of rifaximin provides improved short and long-term survival outcomes for patients suffering from overt HE. This finding is significant relative other HE therapies, such as, for example, administration of lactulose either alone or in combination with rifaximin. Furthermore, it was found that hospitalizations are less frequent, and time to hospitalization readmission longer, in patients suffering from HE who were administered a rifaximin monotherapy.

Accordingly, embodiments are directed to a method of increasing the long-term survival outcome for a subject suffering from HE, comprising administering a composition comprising rifaximin to the subject.

Embodiments are also directed to a method of reducing the frequency of hospitalization visits by a subject suffering from HE, comprising administering a composition comprising rifaximin to the subject.

Embodiments also relate to a method of increasing the length of time to hospitalization readmission for a subject suffering from HE, comprising administering a composition comprising rifaximin to the subject.

Embodiments are also directed to a method of decreasing the risk of developing sarcopenia in a subject suffering from cirrhosis, comprising administering a composition comprising rifaximin to the subject. In some embodiments, the subject is also suffering from HE or subclinical hepatic encephalopathy.

Embodiments also provide a method of maintaining at least about 75% of normal daily caloric intake in a subject suffering from cirrhosis, comprising administering a composition comprising rifaximin to the subject. In some embodiments, the subject is also suffering from HE or subclinical hepatic encephalopathy.

Embodiments also relate to a method of decreasing the risk of suffering from malnutrition in a subject suffering from cirrhosis, comprising administering a composition comprising rifaximin to the subject. In some embodiments, the subject is also suffering from HE or subclinical hepatic encephalopathy.

In some embodiments, the subject is not administered lactulose. In some embodiments, the subject is not concurrently administered lactulose.

Embodiments are also directed to a method of treating hepatic encephalopathy (HE) by administering a therapeutically effective amount of rifaximin to a subject.

In some embodiments, the measure of improvement in HE in a subject suffering therefrom, or the efficacy of treatments described herein, is provided as a measurement of the time to a first HE-related hospitalization. In some embodiments, the measure of improvement or efficacy of treatment is provided as the time to development of spontaneous bacterial peritonitis (SBP). In some embodiments, the measure of improvement or efficacy of treatment is provided as a mean change from baseline in blood ammonia concentration over time. In some embodiments, the measure of improvement or efficacy of treatment is provided as a change in mean daily lactulose consumption over time, a shift from baseline in Conn scores over time; or a shift from baseline in asterixis grades over time. Unless otherwise specified, a shift of a value is the change of that value from a baseline value.

Other measures of efficacy of the treatments described herein included mean change from baseline in Chronic Liver Disease Questionnaire (CLDQ) scores over time; mean change from baseline in Epworth Sleepiness Scale scores over time; and proportion of subjects who have an Epworth Sleepiness Scale score >10. The evaluation of severity of persistent hepatic encephalopathy may also be based, for example, on Conn scores.

Toxic compounds can gain access to the systemic circulation as a result of decreased hepatic function or portal-systemic shunts. Once in brain tissue, the compounds produce alterations of neurotransmission that affect consciousness and behavior. HE is attributed to global central nervous system depression from nitrogenous compounds that result in excitation of gamma-aminobutyric acid (GABA) and decreased neurotransmission of glutamate.

Precipitating factors include azotemia (29%), sedatives, tranquilizers, analgesics (24%), gastrointestinal bleeding (18%), excess dietary protein (9%), metabolic alkalosis (11%), infection (3%), constipation (3%). Surgery, particularly transjugular intrahepatic portal-systemic shunt (TIPS) procedures, also may precipitate HE. HE due to unknown causes accounts for only 2% of cases.

Initial manifestations are subclinical and require psychometric testing for diagnosis. There are 4 progressive stages of impairment known as the West Haven criteria (or Conn score) which range from Stage 0 (Lack of detectable changes in personality) to Stage 4 (Coma, decerebrate posturing, dilated pupils) as discussed in more detail below.

HE is manifested as a continuum of psychomotor dysfunction, impaired memory, increased reaction time, sensory abnormalities, poor concentration and in severe forms, as coma. Changes may be observed in personality, consciousness, behavior and neuromuscular function. Neurologic signs may include hyperreflexia, rigidity, myoclonus and asterixis (coarse “flapping” muscle tremor). Cognitive tasks such as connecting numbers with lines can be abnormal. Fetor hepaticus (sweet breath odor) may be present. Electroencephalogram (EEG) tracings show nonspecific slow, triphasic wave activity mainly over the frontal areas. Prothrombin time may be prolonged and not correctable with Vitamin K. A computed tomography scan of the head may be normal or show general atrophy. Finally, signs of liver disease such as jaundice and ascites may be noted.

Diagnosis of HE is made on the basis of medical history, and physical and mental status examinations with the required clinical elements being knowledge of existent liver disease, precipitating factor(s), and/or prior history of HE. An EEG may show slow-wave activity, even in mild cases. An elevated serum ammonia level is characteristic but not essential, and correlates poorly with the level of encephalopathy

Management of patients with chronic HE includes 1) provision of supportive care, 2) identification and removal of precipitating factors, 3) reduction of nitrogenous load from the gut, and 4) assessment of the need for long term therapy. The nitrogenous load from the gut is typically reduced using non-absorbable disaccharide (lactulose) and/or antibiotics.

Lactulose is considered a first-line treatment in the United States. Lactulose is metabolized by the intestinal bacteria of the colon, which leads to reduced fecal pH, then to a laxative effect, and finally to fecal elimination. The reduced fecal pH ionizes ammonia (NH₃) to the ammonium ion (NH₄⁺) which is used by the bacteria for amino acid and protein synthesis. This lowers the serum ammonia levels and improves mental function.

Conventional therapy aims to lower the production and absorption of ammonia. Lactulose is typically used in doses of 30-60 g daily. However, the dose can be titrated up to 20-40 g TID-QID to affect 2-3 semi-formed bowel movements per day. If lactulose cannot be administered orally or per nasogastric tube, for example to patients with stage 3 and 4 HE, it may be given as a 300 cc (200 g) retention enema.

For acute encephalopathy, lactulose can be administered either orally, by mouth or through a nasogastric tube, or via retention enemas. The usual oral dose is 30 g followed by dosing every 1 to 2 hours until evacuation occurs. At that point, dosing is adjusted to attain two or three soft bowel movements daily.

Lactulose for is readily available over-the-counter. A convenient and relatively tasteless formulation, often referred to in the trade as “lactulose powder for oral solution” can be obtained, for example, from Bertek Pharmaceuticals, Sugarland, Tex. as Kristalose® in 10 and 20 gm packets. The lactulose syrups commonly sold as laxatives include Cephulac®, Chronulac®, Cholac®, and Enulose®. These syrups can be substituted for lactulose powder by using sufficient syrup to provide the desired dosage of lactulose; typically, the named syrups contain about 10 gm lactulose in 15 ml of syrup.

Rifaximin is a nonaminoglycoside, semisynthetic antibiotic derived from rifamycin O. It is a non-systemic, non-absorbed, broad-spectrum, oral antibiotic specific for enteric pathogens of the GI tract. Rifaximin was found to be advantageous in treatment of HE relative to previously used antibiotics; e.g., negligible systemic absorption (<0.4%) regardless of food intake or presence of GI disease and exhibits no plasma accumulation with high or repeat doses. The lack of systemic absorption makes rifaximin safe and well tolerated, thus improving patient compliance and reducing side effects associated with currently known treatments.

Rifaximin (INN; see The Merck Index, XIII Ed., 8304) is an antibiotic belonging to the rifamycin class of antibiotics, e.g., a pyrido-imidazo rifamycin. Rifaximin exerts its broad antibacterial activity, for example, in the gastrointestinal tract against localized gastrointestinal bacteria that cause infectious diarrhea, irritable bowel syndrome, small intestinal bacterial overgrowth, Crohn's disease, and/or pancreatic insufficiency. It has been reported that rifaximin is characterized by a negligible systemic absorption, due to its chemical and physical characteristics (Descombe J. J. et al. Pharmacokinetic study of rifaximin after oral administration in healthy volunteers. Int J Clin Pharmacol Res, 14 (2), 51-56, (1994)).

Rifaximin is described in Italian Patent IT 1154655 and EP 0161534. EP patent 0161534 discloses a process for rifaximin production using rifamycin 0 as the starting material (The Merck Index, XIII Ed., 8301). U.S. Pat. No. 7,045,620 B1 discloses polymorphic forms of rifaximin. The applications and patents referred to here are incorporated herein by reference in their entirety for all purposes

A rifamycin class antibiotic is, for example, a compound having the structure of Formula I:

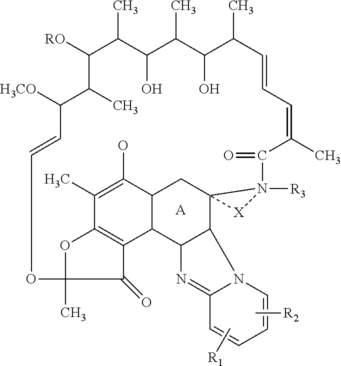

wherein A may be the structure A₁:

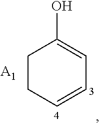

or the structure A₂

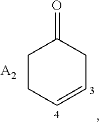

wherein, -x- is a covalent chemical bond or nil; R is hydrogen or acetyl;

R₁ and R₂ independently represent hydrogen, (C₁₋₄) alkyl, benzyloxy, mono- and di-(C₁₋₃) alkylamino-(C₁₋₄) alkyl, (C₁₋₃)alkoxy-(C₁₋₄)alkyl, hydroxymethyl, hydroxy-(C₂₋₄)-alkyl, nitro or R₁ and R₂ taken together with two consecutive carbon atoms of the pyridine nucleus form a benzene ring unsubstituted or substituted by one or two methyl or ethyl groups; R₃ is a hydrogen atom or nil; with the proviso that, when A is A₁, -x- is nil and R₃ is a hydrogen atom; with the further proviso that, when A is A₂, -x- is a covalent chemical bond and R₃ is nil.

Also described herein is a compound as defined above, wherein A is A₁ or A₂ as above indicated, -x- is a covalent chemical bond or nil, R is hydrogen or acetyl, R₁ and R₂ independently represent hydrogen, (C₁₋₄)alkyl, benzyloxy, hydroxy-(C₂₋₄) alkyl, di-(C₁₋₃) alkylamino-(C₁₋₄) alkyl, nitro or R₁ and R₂ taken together with two consecutive carbon atoms of the pyridine nucleus form a benzene ring and R₃ is a hydrogen atom or nil; with the proviso that, when A is A₁, -x- is nil and R₃ is a hydrogen atom; with the further proviso that, when A is A₂, -x- is a covalent chemical bond and R₃ is nil.

Also described herein is a compound as defined above, wherein A is A₁ or A₂ as above indicated, -x- is a covalent chemical bond or nil, R is acetyl, R₁ and R₂ independently represent hydrogen, (C₁₋₄) alkyl or R₁ and R₂ taken together with two consecutive carbon atoms of the pyridine nucleus form a benzene ring and R₃ is a hydrogen atom or nil; with the proviso that, when A is A₁, -x- is nil and R₃ is a hydrogen atom; with the further proviso that, when A is A₂, -x- is a covalent chemical bond and R₃ is nil.

Also described herein is a compound as defined above, which is 4-deoxy-4′-methyl-pyrido[1′,2′-1,2]imidazo[5,4-c]rifamycin SV. Also described herein is a compound as defined above, which is 4-deoxy-pyrido[1′,2′:1,2]imidazo[5,4-c] rifamycin SV.

Also described herein is a compound as defined above, wherein A is as described above, -x- is a covalent chemical bond or nil; R is hydrogen or acetyl; R₁ and R₂ independently represent hydrogen, (C₁₋₄) alkyl, benzyloxy, mono- and di-(C₁₋₃alkylamino(C₁₋₄)alkyl, (C₁₋₃)alkoxy-(C₁₋₄)alkyl, hydroxymethyl, hydroxy-(C₂₋₄)-alkyl, nitro or R₁ and R₂ taken together with two consecutive carbon atoms of the pyridine nucleus form a benzene ring unsubstituted or substituted by one or two methyl or ethyl groups; R₃ is a hydrogen atom or nil; with the proviso that, when A is A₁, -x- is nil and R₃ is a hydrogen atom; with the further proviso that, when A is A₂, -x- is a covalent chemical bond and R₃ is nil.

Rifaximin is a compound having the structure of formula II:

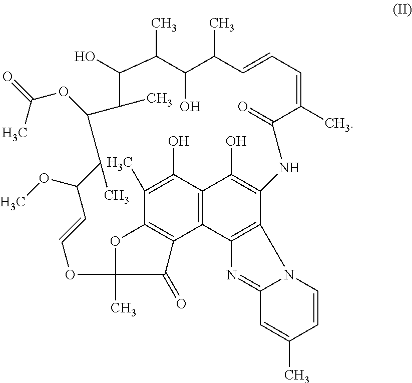 [SMILES: COC1/C=C/OC2(C)Oc3c(C)c(O)c4c(O)c(c5c(nc6cc(C)ccn65)c4c3C2=O)NC(=O)/C(C)=C\C=C\C(C)C(O)C(C)C(O)C(C)C(OC(C)=O)C1C]

In certain embodiments, the antibiotic comprises one or more of a rifamycin, aminoglycoside, amphenicol, ansamycin, β-Lactam, carbapenem, cephalosporin, cephamycin, monobactam, oxacephem, lincosamide, macrolide, polypeptide, tetracycline, or a 2,4-diaminopyrimidine class antibiotic. Exemplary antibiotics of these classes are listed below.

Rifaximin exerts a broad antibacterial activity in the gastrointestinal tract against localized gastrointestinal bacteria that cause infectious diarrhea, including anaerobic strains. It has been reported that rifaximin is characterized by a negligible systemic absorption, due to its chemical and physical characteristics (Descombe J. J. et al. Pharmacokinetic study of rifaximin after oral administration in healthy volunteers. Int J Clin Pharmacol Res, 14 (2), 51-56, (1994)).

Without wishing to be bound by any particular scientific theories, rifaximin acts by binding to the beta-subunit of the bacterial deoxyribonucleic acid-dependent ribonucleic acid (RNA) polymerase, resulting in inhibition of bacterial RNA synthesis. It is active against numerous gram (+) and (−) bacteria, both aerobic and anaerobic. In vitro data indicate rifaximin is active against species of Staphylococcus, Streptococcus, Enterococcus, and Enterobacteriaceae. Bacterial reduction or an increase in antimicrobial resistance in the colonic flora does not frequently occur and does not have a clinical importance. Rifaximin is currently approved in 17 countries outside the US and was licensed by the Food and Drug Administration (FDA) for the US in May 2004.

It is to be understood that both the foregoing general description and the following detailed description are exemplary and explanatory only and are not restrictive of the invention as claimed. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

All documents, or portions of documents, cited in this application, including but not limited to patents, patent applications, articles, books, and treatises, are hereby expressly incorporated by reference in their entirety for any purpose.

In some embodiments, a subject suffering from, susceptible to or in remission from hepatic encephalopathy (HE) can be administered rifaximin for between about 12 weeks and 36 months. In treating HE, the rifamycin class antibiotic may be administered daily to the subject for 6 months and longer, for example, for at least about six months, 12 months, 24 months, 36 months, or for a subject's entire life span or the duration of a subject's life. In some embodiments, the antibiotic is administered daily until the death of the subject.

In some embodiments, a therapeutically effective amount of rifaximin comprises from between about 50 mg to about 6000 mg/day.

In some embodiments, the therapeutically effective amount of rifaximin comprises from between about 1000 mg to about 1200 mg/day.

In some embodiments, the therapeutically effective amount of rifaximin comprises from between about 1100 mg to about 1200 mg/day.

In some embodiments, the therapeutically effective amount of rifaximin comprises a dose starting about 1200 mg/day before being subsequently reduced to a dose of about 1100 mg/day.

In some embodiments, the therapeutically effective amount of rifaximin comprises about 1150 mg/day.

In some embodiments, the therapeutically effective amount is a dosage regimen of one capsule or tablet of the formulation two times each day, wherein each tablet comprises about 550 mg of rifaximin.

In some embodiments, the therapeutically effective amount is a dosage regimen of two capsules or tablets three times each day, wherein each capsule comprises about 200 mg of rifaximin.

In some embodiments, the therapeutically effective amount is a dosage of 275 mg of rifaximin administered four times per day. In some embodiments, 275 mg of rifaximin is administered as two dosage forms two times per day.

Embodiments also relate to a method of reducing the frequency of hospitalization visit by a subject suffering from HE, comprising administering to the subject rifaximin. In some embodiments, the administration of rifaximin reduces hospitalization frequency by about 48%. In some embodiments, rifaximin reduces hospitalization frequency by from between about 13% to about 69%.

Embodiments are also directed to a method of increasing time to hospitalization for treatment of HE in a subject, comprising comprising administering to the subject rifaximin. In some embodiments, the method increases the length of time to hospital readmission for the subject.

In some embodiments, rifaximin is administered to the subject for six months, one year, two to three years or daily until the subject's death.

In some embodiments, a Conn score for the subject is improved over baseline following administration of rifaximin.

In some embodiments, a quality of life (QoL) measurement is improved from baseline with administration of a course of treatment with rifaximin. In one embodiment, the improvised quality is an improvement in the AUC or TWA of the Chronic Liver Disease Questionnaire (CLDQ).

In some embodiments, rifaximin is administered to the subject with lactulose, prior to treatment with lactulose, or following treatment with lactulose. In some embodiments, the subject or a health care worker is advised to administer rifaximin with lactulose. In some embodiments, the subject or a health care worker is advised by a pharmaceutical label or insert to administer rifaximin with lactulose in order to maintain remission of HE, or to decrease the risk for episodes of overt HE. In some embodiments, the subject or health care worker is advised to administer two 550 mg tablets of rifaximin twice daily with lactulose. Lactulose use may be titrated over time so that the subject maintains 2-3 soft stool bowel movements per day. In some embodiments, the lactulose is administered in 15 ml dosages, wherein each 15 ml dosage contains 10 mg of lactulose. In a typical titration, the subject may start on one dosage, or a partial dosage, per day and then move up in 15 ml dosages over time until they reach an end point of 2-3 soft stool bowel movements per day.

In some embodiments, rifaximin is administered to the subject without lactulose.

In some embodiments, subjects in need of treatment for HE and having a Child-Pugh grade of A, B or C are treated with rifaximin. In some embodiments, subjects in need of treatment for HE having a Child-Pugh grade of A, BB or C are treated with rifaximin in combination with lactulose. In some embodiments, subjects having a Child-Pugh grade of A, B, or C, or their health care worker, are advised that they should be treated with rifaximin. The advice can be oral or written advice, such as on a pharmaceutical label or package insert. In some embodiments, subjects having a Child-Pugh grade of A, B or C, or their health care worker, are advised that they should be treated with rifaximin alone or in combination with lactulose. In some embodiments, a subject in need of treatment for HE and having a Child-Pugh grade of less than C is treated with rifaximin. In some embodiments, a subject in need of treatment for HE and having a Child-Pugh grade of less than C is treated with rifaximin and lactulose. In some embodiments, a subject in need of treatment for HE and having a Child-Pugh grade of at least C is treated with rifaximin. In some embodiments, a subject in need of treatment for HE and having a Child-Pugh grade of at least C is treated with rifaximin and lactulose.

In some embodiments, a subject in need of treatment for HE, or their health care worker is advised of the risk for anaphylaxis prior to treatment with rifaximin.

In some embodiments, rifaximin is administered with one or more of align, alinia, Lactulose, pentasa, cholestyramine, sandostatin, vancomycin, lactose, amitiza, flagyl, zegerid, prevacid, or miralax.

In some embodiments, following treatment with rifaximin, a Conn score (mental state grade) of a subject decreases.

In some embodiments, following treatment with rifaximin, a Conn score increase from baseline is increased.

In some embodiments, following treatment with rifaximin, a delay in time to an increase in Conn score is about 54%. For example, the percentage delay in time to increase in Conn score may be between about 30% to about 70%.

In some embodiments, administration of rifaximin prevents an increase in Conn score. For example, administration of rifaximin increases the time to an increase from baseline in a Conn score.

In some embodiments, administration of rifaximin results in an increase of time to an increase from baseline in an asterixis grade.

In some embodiments, administration of rifaximin results in a delay in the time to increase in asterixis grade.

In some embodiments, administration of rifaximin results in an increase in time to first HE-related hospitalization.

In some embodiments, administration of rifaximin results in an increase in time to hospital readmission.

In some embodiments, administration of rifaximin results in an increase in the time to development of spontaneous bacterial peritonitis (SBP).

In some embodiments, administration of rifaximin results in a decrease in blood ammonia concentration from baseline after administration of rifaximin. For example, the decrease in blood ammonia concentration may be from baseline to 170 days of about 6 μg/dL.

In some embodiments, administration of rifaximin results in a decrease in daily lactulose consumption from baseline over time after administration with rifaximin.

In some embodiments, administration of rifaximin results in a decrease in daily lactulose consumption is from between about 7 doses of lactulose to about 2 doses of lactulose.

In some embodiments, administration of rifaximin results in a lactulose use that initially increases from baseline. For example, the lactulose use may be from between about 1 and about 30 days.

In some embodiments, administration of rifaximin results in a shift in baseline in Conn scores over time after administration of rifaximin. For example, the shift in baseline in Conn scores may be from between about 1 to about 2.

In some embodiments, administration of rifaximin results in a shift from baseline in asterixis grades over time.

In some embodiments, administration of rifaximin results in a change from baseline in Chronic Liver Disease Questionnaire (CLDQ) scores over time.

In some embodiments, administration of rifaximin results in a change from baseline in Epworth Sleepiness Scale scores over time after administration of rifaximin.

As is known, the Model for End-Stage Liver Disease (MELD) score can be utilized to predict liver disease severity based on serum creatinine, serum total bilirubin, and the international normalized ratio for prothrombin time INR. The MELD score and has been shown to be useful in predicting mortality in patients with compensated and decompensated cirrhosis. The maximum score given for MELD is 40. All values higher than 40 are given a score of 40.

In some embodiments, the subject to which rifaximin is administered has a MELD score of at least about 25.

In some embodiments, subjects having a MELD level of between about 1 to 24 responded to treatment for HE using administration of the GI specific. In another embodiment, subjects having a MELD level less than or equal to 10 responded to treatment with rifaximin. In another embodiment, subjects having a MELD level between 11 and 18 respond to treatment with Rifaximin. In another embodiment, subjects having a MELD level between 19 and 24 respond to treatment with Rifaximin. In one embodiment, subjects in need of treatment for HE and having a MELD score of 25 or less are treated with rifaximin. In another embodiment, subjects in need of treatment for HE having a MELD score of 25 or less are treated with rifaximin in combination with lactulose. In another embodiment, subjects having a MELD score of 25 or less are advised that they should be treated with rifaximin. The advice can be oral or written advise, such as on a pharmaceutical label or package insert. In another embodiment, subjects having a MELD score of 25 or less are advised that they should be treated with rifaximinin combination with lactulose.

One embodiment presented herein is a method of treating or preventing HE by administering 1100 mg of rifaximin per day to a patient for more than 28 days.

Another embodiment is a method of decreasing lactulose use in a subject. This method includes: administering rifaximin to a subject daily that is being treated with lactulose, and tapering lactulose consumption. For example, the lactulose consumption may be reduced by 1, 2, 3, 4, 5, 6 or more unit dose cups of lactulose from a baseline level. Alternatively, the lactulose use may be reduced by 5, 10, 15, 20, 25, 30, 34, 40, 45, 50, 55, 60, 65, or 70 g lactulose from a baseline level. In one embodiment, the baseline use of lactulose is no use.

One embodiment presented herein is a method of maintaining remission of HE in a subject comprising administering 550 mg of rifaximin twice a day (BID) to the subject.

In some embodiments, a method of increasing the long-term survival of a subject suffering from HE is provided, comprising administering to a subject a therapeutically effective amount of rifaximin. In some embodiments, the therapeutically effective amount of rifaximin is about 1100 mg to about 1200 mg/day. In some embodiments, the therapeutically effective amount of rifaximin is provided as a dosage of 550 mg administered two times per day (BID). In some embodiments, the therapeutically effective amount of rifaximin is provided as a dosage of 400 mg administered three times per day (TID). In some embodiments, the rifaximin is administered daily for at least about six months. In some embodiments, the rifaximin is administered daily for at least about 12 months. In some embodiments, the rifaximin is administered daily for at least about 24 months. In some embodiments, the rifaximin is administered daily for at least about 36 months. In some embodiments, the rifaximin is administered daily for the duration of the subject's life.

Another embodiment is a method of increasing time to hospitalization for treatment of HE comprising, administering to a subject 550 mg of rifaximin two times per day (BID).

In some embodiments, a method of increasing time to hospital readmission for a subject suffering from HE is provided, comprising administering to a subject a therapeutically effective amount of rifaximin. In some embodiments, the therapeutically effective amount of rifaximin is about 1100 mg to about 1200 mg/day. In some embodiments, the therapeutically effective amount of rifaximin is provided as a dosage of 550 mg administered two times per day (BID). In some embodiments, the therapeutically effective amount of rifaximin is provided as a dosage of 400 mg administered three times per day (TID).

The term “administration” or “administering” includes routes of introducing rifaximin to a subject to perform their intended function. Examples of routes of administration that may be used include injection (subcutaneous, intravenous, parenterally, intraperitoneally, intrathecal), oral, inhalation, rectal and transdermal. The pharmaceutical preparations may be given by forms suitable for each administration route. For example, these preparations are administered in tablets or capsule form, by injection, inhalation, eye lotion, eye drops, ointment, suppository, etc. administration by injection, infusion or inhalation; topical by lotion or ointment; and rectal by suppositories. Oral administration is preferred. The injection can be bolus or can be continuous infusion. Depending on the route of administration, a GI specific antibiotic can be coated with or disposed in a selected material to protect it from natural conditions that may detrimentally effect its ability to perform its intended function. A GI specific antibiotic can be administered alone, or in conjunction with either another agent or agents as described above or with a pharmaceutically-acceptable carrier, or both. A GI specific antibiotic can be administered prior to the administration of the other agent, simultaneously with the agent, or after the administration of the agent. Furthermore, a GI specific antibiotic can also be administered in a proform, which is converted into its active metabolite, or more active metabolite in vivo.

Administration “in combination with” one or more further therapeutic agents includes simultaneous (concurrent) and consecutive administration in any order.

As will be readily apparent to one skilled in the art, the useful in vivo dosage to be administered and the particular mode of administration will vary depending upon the age, weight and mammalian species treated, the particular compounds employed, and the specific use for which these compounds are employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved.

As used herein, an “increase” or “decrease” in a measurement, unless otherwise specified, is typically in comparison to a baseline value. For example, an increase in time to hospitalization for subjects undergoing treatment may be in comparison to a baseline value of time to hospitalization for subjects that are not undergoing such treatment. In some instances an increase or decrease in a measurement can be evaluated based on the context in which the term is used.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN, polyethylene glycol (PEG).

The term “effective amount” includes an amount effective, at dosages and for periods of time necessary, to achieve the desired result, e.g., sufficient to treat HE in a patient or subject. An effective amount of rifaximin may vary according to factors such as the disease state, age, and weight of the subject, and the ability of rifaximin to elicit a desired response in the subject. Dosage regimens may be adjusted to provide the optimum therapeutic response. An effective amount is also one in which any toxic or detrimental effects (e.g., side effects) of rifaximin are outweighed by the therapeutically beneficial effects.

“Ameliorate,” “amelioration,” “improvement” or the like refers to, for example, a detectable improvement or a detectable change consistent with improvement that occurs in a subject or in at least a minority of subjects, e.g., in at least about 2%, 5%, 10%, 15%, 20%, 25%, 30%, 40%, 50%, 60%, 70%, 75%, 80%, 85%, 90%, 95%, 98%, 100% or in a range between about any two of these values. Such improvement or change may be observed in treated subjects as compared to subjects not treated with rifaximin, where the untreated subjects have, or are subject to developing, the same or similar disease, condition, symptom or the like. Amelioration of a disease, condition, symptom or assay parameter may be determined subjectively or objectively, e.g., self assessment by a subject(s), by a clinician's assessment or by conducting an appropriate assay or measurement, including, e.g., a quality of life assessment such as a Chronic Liver Disease Questionnaire (CLDQ), a slowed progression of a disease(s) or condition(s), a reduced severity of a disease(s) or condition(s), or a suitable assay(s) for the level or activity(ies) of a biomolecule(s), cell(s) or by detection of HE episodes in a subject. Amelioration may be transient, prolonged or permanent or it may be variable at relevant times during or after rifaximin is administered to a subject or is used in an assay or other method described herein or a cited reference, e.g., within timeframes described infra, or about 1 hour after the administration or use of rifaximin to about 28 days, or 1, 3, 6, 9 months or more after a subject(s) has received such treatment.

The “modulation” of, e.g., a symptom, level or biological activity of a molecule, or the like, refers, for example, that the symptom or activity, or the like is detectably increased or decreased. Such increase or decrease may be observed in treated subjects as compared to subjects not treated with rifaximin, where the untreated subjects have, or are subject to developing, the same or similar disease, condition, symptom or the like. Such increases or decreases may be at least about 2%, 5%, 10%, 15%, 20%, 25%, 30%, 40%, 50%, 60%, 70%, 75%, 80%, 85%, 90%, 95%, 98%, 100%, 150%, 200%, 250%, 300%, 400%, 500%, 1000% or more or within any range between any two of these values. Modulation may be determined subjectively or objectively, e.g., by the subject's self assessment, by a clinician's assessment or by conducting an appropriate assay or measurement, including, e.g., quality of life assessments or suitable assays for the level or activity of molecules, cells or cell migration within a subject. Modulation may be transient, prolonged or permanent or it may be variable at relevant times during or after rifaximin is administered to a subject or is used in an assay or other method described herein or a cited reference, e.g., within times descried infra, or about 1 hour of the administration or use of rifaximin to about 3, 6, 9 months or more after a subject(s) has received rifaximin.

The term “modulate” may also refer to increases or decreases in the activity of a cell in response to exposure to rifaximin, e.g., the inhibition of proliferation and/or induction of differentiation of at least a sub-population of cells in an animal such that a desired end result is achieved, e.g., a therapeutic result of rifaximin used for treatment may increase or decrease over the course of a particular treatment.

The term “obtaining” as in “obtaining rifaximin” is intended to include purchasing, synthesizing or otherwise acquiring rifaximin.

The phrases “parenteral administration” and “administered parenterally” as used herein includes, for example, modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

Embodiments also provide pharmaceutical compositions, comprising an effective amount of a rifaximin described herein and a pharmaceutically acceptable carrier. In a further embodiment, the effective amount is effective to treat hepatic encephalopathy in a subject suffering from hepatic insufficiency.

Embodiments also provide pharmaceutical compositions comprising rifaximin and a pharmaceutically acceptable carrier. Doses may be selected, for example on the basis of desired amounts of systemic adsorption, elimination half-life, serum concentration and the like. Embodiments of the pharmaceutical composition further comprise excipients, for example, one or more of a diluting agent, binding agent, lubricating agent, disintegrating agent, coloring agent, flavoring agent or sweetening agent. One composition may be formulated for selected coated and uncoated tablets, hard and soft gelatin capsules, sugar-coated pills, lozenges, wafer sheets, pellets and powders in sealed packet. For example, compositions may be formulated for topical use, for example, ointments, pomades, creams, gels and lotions.

In an embodiment, rifaximin is administered to the subject using a pharmaceutically-acceptable formulation, e.g., a pharmaceutically-acceptable formulation that provides sustained delivery of the rifaximin to a subject for at least 12 hours, 24 hours, 36 hours, 48 hours, one week, two weeks, three weeks, or four weeks after the pharmaceutically-acceptable formulation is administered to the subject.

In certain embodiments, these pharmaceutical compositions are suitable for topical or oral administration to a subject. In other embodiments, as described in detail below, the pharmaceutical compositions presented herein may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “pharmaceutically acceptable” refers to rifaximin compositions containing rifaximin and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” includes pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier is preferably “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions containing a rifaximin forms disclosed herein include those suitable for oral, nasal, topical (including buccal and sublingual), rectal, vaginal, aerosol and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred %, this amount will range from about 1% to about ninety-nine % of active ingredient, preferably from about 5% to about 70%, most preferably from about 10% to about 30%.

Methods of preparing these compositions include the step of bringing into association a rifaximin with the carrier and, optionally, one or more accessory ingredients. In general, the formulations are prepared by uniformly and intimately bringing into association a rifaximin with liquid carriers, or finely divided solid carriers, or both, and then, if necessary, shaping the product.

Compositions suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a rifaximin as an active ingredient. A compound may also be administered as a bolus, electuary or paste.

The term “pharmaceutical agent composition” (or agent or drug) as used herein refers to a chemical compound, composition, agent or drug capable of inducing a desired therapeutic effect when properly administered to a patient. It does not necessarily require more than one type of ingredient.

The compositions may be in the form of tablets, capsules, powders, granules, lozenges, liquid or gel preparations. Tablets and capsules for oral administration may be in a form suitable for unit dose presentation and may contain conventional excipients. Examples of these are: binding agents such as syrup, acacia, gelatin, sorbitol, tragacanth, and polyvinylpyrrolidone; fillers such as lactose, sugar, maize-starch, calcium phosphate, sorbitol or glycine; tableting lubricants, such as magnesium stearate, silicon dioxide, talc, polyethylene glycol or silica; disintegrants, such as potato starch; or acceptable wetting agents, such as sodium lauryl sulfate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, e.g., sorbitol, syrup, methyl cellulose, glucose syrup, gelatin, hydrogenated edible fats, emulsifying agents, e.g., lecithin, sorbitan monooleate, or acacia; non-aqueous vehicles (including edible oils), e.g., almond oil, fractionated coconut oil, oily esters such as glycerine, propylene glycol, or ethyl alcohol; preservatives such as methyl or propyl p-hydroxybenzoate or sorbic acid, and, if desired, conventional flavoring or coloring agents.

The phrases “systemic administration,” “administered systemically,” “peripheral administration,” and “administered peripherally,” as used herein mean the administration of rifaximin, such that it enters the subject's system and, thus, is subject to metabolism and other like processes, for example, subcutaneous administration.

The language “therapeutically effective amount” of rifaximin refers to an amount of rifaximinwhich is effective, upon single or multiple dose administration to the subject, in inhibiting the bacterial growth and/or invasion, or in decreasing symptoms, such as HE episodes, relating to bacterial growth in a subject. “Therapeutically effective amount” also refers to the amount of a therapy (e.g., a composition comprising rifaximin), which is sufficient to reduce the severity of HE in a subject.

As used herein, the term “prophylactically effective amount” refers to the amount of a therapy (e.g., a composition comprising rifaximin) which is sufficient to result in the prevention of the development, recurrence, or onset of HE episodes or to enhance or improve the prophylactic effect(s) of another therapy.

“Rifaximin”, as used herein, includes solvates and polymorphous forms of the molecule, including, for example, Form α, Form β, Form γ Form δ, Form ε, Form ζ, Form η, Form i, Form kappa, Form theta, Form mu, Form omicron, Form pi, mesylate Form or amorphous Forms of rifaximin. These forms are described in more detail, for example, in EP 05 004 695.2, filed 3 Mar. 2005; U.S. Pat. No. 7,045,620; U.S. Pat. No. 7,612,199; U.S. Pat. No. 7,709,634; U.S. Pat. No. 7,915,275; U.S. Pat. No. 8,067,429; U.S. Pat. No. 8,193,196; U.S. Pat. No. 8,227,482; U.S. Pat. No. 8,383,151; U.S. Pat. No. 8,486,956; U.S. Pat. No. 8,513,275; U.S. Pat. No. 8,518,949; G. C. Viscomi, et al., CrystEngComm, 2008, 10, 1074-1081 (April 2008), and US Patent Publication 2005/0272754. Each of these references is hereby incorporated by reference in entirety.

The forms of rifaximin can be advantageously used in the production of medicinal preparations having antibiotic activity, containing rifaximin, for both oral and topical use. The medicinal preparations for oral use may contain one or more forms of rifaximin together with other excipients, for example diluting agents such as mannitol, lactose and sorbitol; binding agents such as starchs, gelatines, sugars, cellulose derivatives, natural gums and polyvinylpyrrolidone; lubricating agents such as talc, stearates, hydrogenated vegetable oils, polyethylenglycol and colloidal silicon dioxide; disintegrating agents such as starchs, celluloses, alginates, gums and reticulated polymers; coloring, flavoring and sweetening agents.

Medicinal preparations may contain gastrointestinal specific antibiotics together with usual excipients, such as white petrolatum, white wax, lanoline and derivatives thereof, stearylic alcohol, red iron oxide, propylene glycol, talc, sodium lauryl sulfate, ethers of fatty polyoxyethylene alcohols, disodium edentate, glycerol palmitostearate, esters of fatty polyoxyethylene acids, sorbitan monostearate, glyceryl monostearate, propylene glycol monostearate, hypromellose, polyethylene glycols, sodium starch glycolate, methylcellulose, hydroxymethyl propylcellulose, sodium carboxymethylcellulose, microcrystalline cellulose, colloidal aluminium and magnesium silicate, titanium dioxide, propylene glycol, colloidal silicon dioxide, or sodium alginate.

As used herein, “breakthrough HE,” includes, for example, an increase of the Conn score to Grade ≧2 (e.g., 0 or 1 to ≧2) or a Conn and Asterixis score increase of 1 grade each for those subjects that have a baseline Conn score of 0.

As used herein, the term “breakthrough HE event”, is intended to include a marked, clinically significant deterioration in neurological function caused by toxic substances accumulating in the blood that cause a deleterious effect on self care, and often leads to hospitalization. Breakthrough HE event is also defined as an increase of a Conn Score to ≧2 (i.e., 0 or 1 to ≧2) or a Conn score and asterixis grade increase of 1 each for those subjects that have a baseline Conn score of 0.

Methods for determining if a subject has HE or has an increased risk of having a HE breakthrough event can be conducted by, for example, measuring the venous ammonia level in a subject at two or more time points, wherein an increase in the venous ammonia level is indicative that the subject has HE, has an increased chance of an HE breakthrough event, and/or should be treated with rifaximin. In certain embodiments the venous ammonia level is a time weighted average venous ammonia level.

Venous ammonia concentration can be measured using methods that are known to one of skill in the art. The accuracy of ammonia determination is dependent on sample collection. Whole blood is preferred. In one specific method described herein, blood is collect blood from a stasis-free vein into an EDTA evacuated tube. The sample is placed in ice immediately after collecting and mixing. The sample is placed in a cold environment, e.g., on ice, for approximately ten minutes and then centrifuged. The plasma is separated from the sample within fifteen minutes of collection and frozen. Hemolyzed samples should not be used for further analysis.

The frozen sample is subjected to an enzymatic assay to determine the amount of ammonia present in the sample. The sample containing ammonia is mixed with α-ketoglutarate and reduced nicotinamide adenine dinucleotide phosphate (NADPH) to form L-glutamate and NADP and water. The reaction is catalyzed by glutamate dehydrogenase. The results are determined spectrophotometrically by monitoring the decrease in absorbance at 340 nm due to the oxidation of NADPH. This decrease is proportional to the ammonia concentration.

As used herein, “time to a HE-related hospitalization,” includes, for example, the duration between the first dose of rifaximin and the date of an HE-related hospitalization following administration of the first dose of rifaximin.

“Time to hospital readmission,” includes, for example, the duration of time between discharge from the hospital for treatment of HE-related symptoms and subsequent admission to the hospital for treatment of the same.

As used herein, “time to an increase from baseline in the Conn score” includes, for example, the duration between the first dose of rifaximin and the date of first increase in Conn score.

As used herein, “time to an increase from baseline in the asterixis grade”, includes, for example, the duration between the first dose of rifaximin and the date of first increase in asterixis grade.

As used herein, “mean change from baseline in the fatigue domain score of Chronic Liver Disease Questionnaire (CLDQ), at end of treatment (EOT)” is the mean score with a baseline from before the first administration of rifaximin.

As used herein, “mean change from baseline in blood ammonia concentration at EOT,” includes the mean score with a baseline from before the first administration of rifaximin.

As used herein, the “time to diagnosis of spontaneous bacterial peritonitis (SBP),” includes, for example, the duration between the first dose of rifaximin and the date of first episode of SBP.

As used herein, “subject” includes organisms which are capable of suffering from hepatic failure, decreased hepatic function, cirrhosis or hepatic encephalopathy, such as human and non-human animals. Preferred human animals include human subjects. The term “non-human animals” includes, for example, all vertebrates, e.g., mammals, e.g., rodents, e.g., mice, and non-mammals, such as non-human primates, e.g., sheep, dog, cow, chickens, amphibians, reptiles, etc.

The language “a prophylactically effective amount” of a compound refers to an amount of rifaximin as described herein which is effective, upon single or multiple dose administration to the subject, in treating hepatic encephalopathy.

Another embodiment includes articles of manufacture that comprise, for example, a container holding a pharmaceutical composition suitable for oral administration of rifaximin in combination with printed labeling instructions providing a discussion of when a particular dosage form extends remission of HE or prevents or delays future episodes of HE. The dosage can be modified for administration to a subject suffering from HE, or include labeling for administration to a subject suffering from HE. Exemplary dosage forms and administration protocols are described infra. The composition will be contained in any suitable container capable of holding and dispensing the dosage form and which will not significantly interact with the composition and will further be in physical relation with the appropriate labeling. The labeling instructions may be consistent with the methods of treatment as described hereinbefore. The labeling may be associated with the container by any means that maintain a physical proximity of the two, by way of non-limiting example, they may both be contained in a packaging material such as a box or plastic shrink wrap or may be associated with the instructions being bonded to the container such as with glue that does not obscure the labeling instructions or other bonding or holding means.

In some embodiments, the instructions will inform and/or advise a health care worker, prescribing physician, a pharmacist, or a subject that they should advise a patient suffering from hepatic encephalopathy that administration of rifaximin may induce cytochrome P450. In some embodiments, the instructions will inform the subject and/or the healthcare provider that there is an extended time to remission or relapse of subjects that take rifaximin. In some embodiments, the instructions will inform the subject and/or the healthcare worker or provider that rifaximin does not significantly alter the Cₘₐₓ, AUC₀₋ₜ, or AUC₀₋₂₈ of midazolam. In some embodiments, the instructions will inform the subject and/or the healthcare worker or provider that rifaximin does not increase the risk of QT prolongation.

Packaged compositions are also provided, and may comprise a therapeutically effective amount of rifaximin tablets or capsules. Kits are also provided herein, for example, kits for treating HE in a subject. The kits may contain, for example, rifaximin and instructions for use when treating a subject for an HE. The instructions for use may contain prescribing information, dosage information, storage information, and the like.

Kits may include pharmaceutical preparations of rifaximins along with pharmaceutically acceptable solutions, carriers and excipients.

Forms of rifaximin can be advantageously used in the production of medicinal preparations having antibiotic activity, containing rifaximin, for both oral and topical use. The medicinal preparations for oral use may contain one or more forms of rifaximin (for example, α or β or γ) together with other excipients, for example diluting agents such as mannitol, lactose and sorbitol; binding agents such as starchs, gelatines, sugars, cellulose derivatives, natural gums and polyvinylpyrrolidone; lubricating agents such as talc, stearates, hydrogenated vegetable oils, polyethylenglycol and colloidal silicon dioxide; disintegrating agents such as starchs, celluloses, alginates, gums and reticulated polymers; coloring, flavoring and sweetening agents.

Solid preparations of gastrointestinal specific antibiotics administrable by the oral route include for instance coated and uncoated tablets, soft and hard gelatin capsules, sugar-coated pills, lozenges, wafer sheets, pellets and powders in sealed packets.

Medicinal preparations may contain gastrointestinal specific antibiotics together with usual excipients, such as white petrolatum, white wax, lanoline and derivatives thereof, stearylic alcohol, red iron oxide, propylene glycol, talc, sodium lauryl sulfate, ethers of fatty polyoxyethylene alcohols, disodium edentate, glycerol palmitostearate, esters of fatty polyoxyethylene acids, sorbitan monostearate, glyceryl monostearate, propylene glycol monostearate, hypromellose, polyethylene glycols, sodium starch glycolate, methylcellulose, hydroxymethyl propylcellulose, sodium carboxymethylcellulose, microcrystalline cellulose, colloidal aluminium and magnesium silicate, titanium dioxide, propylene glycol, colloidal silicon dioxide, or sodium alginate.

Measurements of change in mental status may be done, for example, by the Conn score (also known as the West Haven score). The Conn score has been widely used as a measure of mental state in HE studies and is based on the criteria of Parsons-Smith as modified by Conn. Asterixis will not be considered when assessing the subject's status using the Conn scoring criteria listed below.

The scale used in the Conn scoring system is provided below.
Grade 0=No personality or behavioral abnormality detected
Grade 1=Trivial lack of awareness, euphoria or anxiety; shortened attention span; impairment of addition or subtraction
Grade 2=Lethargy; disorientation for time; obvious personality change; inappropriate behavior
Grade 3=Somnolence to semi-stupor, responsive to stimuli; confused; gross disorientation; bizarre behavior
Grade 4=Coma; unable to test mental state

HE is defined as a spectrum of neuropsychiatric abnormalities seen in patients with liver dysfunction, diagnosed after routine exclusion of other known neurologic disease. HE is a major complication of liver cirrhosis, affecting 30-45% patients. In 2006, the CDC listed cirrhosis as the 12th leading cause of death by disease in the U.S. HE affects the patient's consciousness, personality, intellect and neuromuscular function, and may range from a minimal disturbance in cognition, to coma. HE, as used herein, comprises, for example, episodic, persistent and minimal HE.

In the gut, enteric bacteria act on nitrogen-containing substrates to generate ammonia. In cirrhosis, ammonia from the intestines bypasses the damaged liver as a result of vascular shunts. This increases blood ammonia, which passes into the brain generating glutamine from the amino acid glutamate. The excess glutamine causes many deleterious effects on brain function; it inhibits neurotransmission, interferes with mitochondrial energy metabolism, and causes swelling of astrocytes.

HE associated with Cirrhosis—the most common by far—is type C. HE Type C is sub-classified into episodic, persistent and minimal categories. Episodic and persistent varieties are clinically readily apparent conditions, and hence are denoted as Overt. Episodic HE presents with impairment in all the neurological functions mentioned above. As the term episodic implies, there are periods between episodes when no distinctive symptoms are seen. Episodes may be precipitated by factors such as constipation, infection, dehydration, GI hemorrhage and certain medications. If the cause is not immediately identified, the episode is referred to as spontaneous.

HE episodes are usually reversible with treatment—but they're often recurring.

HE is a clinical diagnosis made by some tools, including the West Haven, or Conn, Score. In use for about 30 years, The HESA scoring algorithm is a relatively new tool used for accurate assignment of Conn criteria. Neuromuscular dysfunction can be measured by eliciting asterixis, or flapping tremor. Blood ammonia levels are often measured to support the diagnosis. Neurophysiological tests, such as critical flicker frequency and EEG, are potentially very useful to support the clinical findings. The Conn criteria use an increasing grade to associate with increasing neurological impairment, (ranging from 0=no impairment to 4=coma)
Grades 1, 2, and 3 represent an worsening in impairment in:
   Consciousness—ranging from a trivial lack of awareness to somnolence;
   impairment in intellectual ability and alterations in personality
   This assessment can be conducted quickly, requires minimal intervention from the examiner or cooperation from the patient,
   And we often use information from family or caregivers to help gauge the severity of HE episodes when the patient is confused.

While patients with grade 1 HE can be managed at home by a caregiver, any escalation to grade 2 or higher may require hospitalization and even management in intensive care. The Conn criteria use an increasing grade to associate with increasing neurological impairment, (ranging from 0=no impairment to 4=coma). Grades 1, 2, and 3 represent an worsening in impairment in: consciousness; intellectual ability and alterations in personality. This assessment can be conducted quickly, requires minimal intervention from the examiner or cooperation from the patient, information from family or caregivers is often used to gauge the severity of HE episodes.

While patients with grade 1 HE can be managed at home by a caregiver, any escalation to grade 2 or higher may require hospitalization and even management in intensive care.

There is a similar grading system for asterixis. If an HE patient is asked to hold out their hands just so, a jerky so called asterixis or flapping tremor will be observed. The number of beats is counted and scored from zero for none to four for almost continuous flapping. This is a simple test but requires a cooperative and conscious patient.

HE presents a vicious cycle of dysfunction and disability that has a dramatic effect on patients, their families and the healthcare system. Early on, impairments in behavior, personality, intellect and consciousness affect the patient's social and family life and ability to hold employment. As the condition worsens, it impacts capacity for self care, medication compliance, lack of compliance further intensifies HE symptoms and frequency of episodes. As a result, patients may need in-home assistance and often land in the ER or hospital beds. Severe HE can be a life threatening event, but it more commonly devastates the QOL of patients and their families; some caregivers liken the experience to caring for unpredictably episodic Alzheimer's disease. Impact on caregiver is shown in FIG. 18.

In terms of the impact on healthcare, the number of HE discharges more than doubled between 1993 to 2007. Costs increased—from about 13k to 30k per hospitalization. So, the goals for HE Therapy include, for example, bringing acute episodes to quick resolution, and preventing recurrent episodes. To achieve these goals, a safe and effective therapy that is well tolerated is needed for long-term treatment. There are serious limitations to the long-term use of the currently approved therapies. The most common, Lactulose, a non-absorbable disaccharide, targets the gut flora responsible for ammonia production. It exerts its effects mainly by purging, with frequent bowel movements. Lactulose therapy relies on dose self-titration, aim is for 2-3 loose stools a day—unfortunately this goal is often exceeded. At ten unpredictable loose stools per day, leaving home—even for a short walk to the store—may become impossible or embarrassing. Patients go on disability because of Lactulose rather than the HE it was prescribed for. Severe diarrhea can cause dehydration and electrolyte abnormalities that may even precipitate an HE episode. Nausea is not uncommon. Understandably, these factors can lead to poor adherence and limit long term use.

### EXAMPLES

It should be appreciated that embodiments of the invention should not be construed to be limited to the examples, which are now described; rather, the invention should be construed to include any and all applications provided herein and all equivalent variations within the skill of the ordinary artisan.

### Example 1

### Patients Receiving Rifaximin Treatment have Improved Long-Term Survival Outcomes

The medical charts of outpatient and hospitalized patients admitted for overt HE were examined over a 137-month (about an 11.5-year) window. Information collected included gender, age, ethnicity, post discharge HE management protocol, HE hospitalization number (frequency) and time (days) to transplant or death. Four groups were identified: (1) lactulose-treatment only (LAC), (2) lactulose treatment combined with rifaximin treatment (LAC/RFX), (3) rifaximin only treatment (RFX), and (4) no therapy. The rifaximin dose (RFX) was initially reported with 1200 mg per day dosing, which was later changed to 1100 mg/day (550 mg twice per day). The lactulose dose (LAC) was 30-120 cc/day, adjusted to acquire two to three loose to soft bowel movements. Time from discharge to death or transplant was reported.

In the study, 185 patients were identified, 106 of which were males, 79 or which were females. The median age was 48.5 years (range: 28-72 years). The ethnic group breakdown was as follows: 66% Caucasian, 12% African American, 18% Hispanic, 4% Other. The mean MELD score was 18.5 points (range: 12-40).

As indicated in Table 1, the survival outcomes revealed improvement in both short- and long-term survival following the addition of rifaximin, either alone or in combination with lactulose, in patients suffering from overt HE.

TABLE 1
Survival Rates

| Group | Six Months | One Year | Three Years | p Value |
|---|---|---|---|---|
| 1) LAC (n = 58) | 60% | 46% | 29% | |
| 2) LAC/RFX (n = 55) | 65% | 54% | 41% | 0.057 |
| 3) RFX (n = 28) | 76% | 63% | 51% | 0.049 |
| 4) None (n = 44) | 45% | 35% | 12% | |

LAC group vs. LAC/RFX group: p value = 0.057.
LAC group vs. RFX group: p value = 0.049

Accordingly, embodiments are directed to methods of improving the survival outcome for a subject suffering from hepatic encephalopathy, comprising administering a composition comprising rifaximin to the subject.

In some embodiments, the survival outcome of a rifaximin-treated subject is improved by at least about 5% relative to a subject that has not been treated with rifaximin. In some embodiments, the survival outcome of a rifaximin-treated subject is improved by at least about 10% relative to a subject that has not been treated with rifaximin. In some embodiments, the survival outcome of a rifaximin-treated subject is improved by at least about 15%, 20%, 25%, 30%, 35%, 40%, 45%, 50%, 55%, 60%, 65%, 70% or 75% relative to a subject that has not been treated with rifaximin.

In some embodiments, the improvement is observed over a six-month period of time. In some embodiments, the improvement is observed over a one-year period of time. In some embodiments, the improvement is observed over a three-year period of time.

### Example 2

### Management of Cirrhotic Patients Suffering from HE is More Cost Efficient and Requires Less Hospitalization with Rifaximin Monotherapy Treatment

The medical charts of outpatient and hospitalized patients admitted for overt HE were examined over a 137-month (about an 11.5-year) window. Information collected included gender, age, ethnicity, MELD score, post discharge HE management protocol, HE hospitalization number (frequency), time to readmission (days) and health insurance coverage. All patients suffering from infection, renal failure, gastrointestinal bleeding or noncompliance were excluded. Group one consisted of patients discharged with lactulose (LAC) treatment. Group two consisted of patients discharged on rifaximin (RFX) treatment. Group three consisted of patients discharged on rifaximin treatment combined with lactulose treatment (RFX/LAC). The The rifaximin dose (RFX) was 1100 mg/day (550 mg twice per day). The lactulose dose (LAC) was 30-120 cc/day, adjusted to acquire two to three loose to soft bowel movements. Time from discharge to readmission was measured. All patients were required to have a follow up visit after discharge and maintain family support and employment. Patients were broken down into compliant and non-compliant groups with outpatient follow-up.

Eighty-seven (87) patients were identified as compliant, while forty-nine (49) patients were identified as non-compliant. In the compliant group, there were 51 males and 36 females, with an age range of between 32 and 68 years. The ethnic group breakdown for the compliant group was as follows: 66% Caucasian, 14% African American, 17% Hispanic and 3% other. The mean MELD score of the compliant group was 12.5 points (range: 8-17). In the non-compliant group, there were 29 males and 20 females, with an age range of between 28 and 66 years. The ethnic group breakdown for the non-compliant group was as follows: 64% Caucasian, 18% African American and 18% Hispanic. The mean MELD score of the non-compliant group was 11.5 points (range: 6-18).

In both patient populations, Group 1 (LAC) had the highest charges and number of hospital readmissions, with overall cost differences by 46% and 9% relative to those of Group 2 (RFX) and Group 3 (RFX/LAC), respectively. The results indicate that managing cirrhotic patients suffering from HE with rifaximin treatment is nearly 50% more cost efficient than with lactulose monotherapy or combined rifaximin and lactulose therapy. In addition, hospitalizations are less frequent and time to hospital readmission is greater with rifaximin monotherapy relative to those of lactulose monotherapy or combined therapy.

### Example 3

### Malnutrition in Cirrhotic Patients and an Evaluation of Daily Calorie Counts and Nutritional Deficits in the Same

The medical charts were reviewed for non-hospitalized patients with compensated cirrhosis with and without the diagnosis of subclinical hepatic encephalopathy. The patients were stratified into three groups: 1) cirrhotic without SHE, 2) cirrhotic with SHE treated with lactulose (LAC), and 3) cirrhotic with SHE treated with rifaximin (RFX). Treated patients received either RFX (Group 3) or LAC therapy (Group 2) for SHE and were given daily calorie logs. All patients were reported to be at baseline Conn score Grade 0-1 HE, and employment status was identified. Information collected included; gender, age, ethnicity, weight/height, exercise and employment status, and daily calorie counts for 14 days. Daily caloric intake was registered with food consumption logs by patients and family. Normal caloric intake was considered at 1600 to 2200 calories/day depending upon dietary requirements. The daily caloric totals were conducted with the Calorie Smart calculator. The RFX dose was 1100 mg/day (550 mg twice per day) or LAC 30 cc adjusted according to two to three soft bowel movements each day or no therapy.

There were 64 patients involved in the study: 29 females, 35 males, age (range 19-68), ethnicity (81% Caucasian, 8% African American, 8% Hispanic, 3% other), and mean MELD 8.5 points (range 6-13). There were no events of occult HE or hospitalizations throughout the review.

The results in Table 2 demonstrate the challenges with adequate nutritional intake in compensated cirrhotic patients with SHE. Patients taking lactulose suffered from bowel bloating culminating in a sensation of decreased appetite thus a marked decrease in caloric intake. In contrast, patients that were administered rifaximin appeared to maintain daily caloric intake relative to those patients that did not suffer from SHE and did not undergo SHE therapy.

Accordingly, embodiments are directed to methods of maintaining normal daily caloric intake in a subject suffering from cirrhosis and subclinical hepatic encephalopathy, comprising administering a composition comprising rifaximin to the subject. In some embodiments, the subject maintains at least about 75% of normal daily caloric intake. In some embodiments, the subject maintains at least about 80% of normal daily caloric intake. In some embodiments, the subject maintains at least about 85% or at least about 90% of normal daily caloric intake.

In some embodiments, administration of rifaximin reduces the risk of developing sarcopenia in the subject. In some embodiments, administration of rifaximin reduces the risk of suffering from malnutrition in the subject.

TABLE 2
Survival Rates

| | Daily caloric | | | |
|---|---|---|---|---|
| | intake | | Un- | |
| | (normal = 1600- | Employed | employed | |
| Group | 2200/day) | (range) | (range) | p Value |
| 1) No therapy | 75% to 82% | 72% to 80% | 65% to 78% | |
| (n = 20) | | | | |
| 2) LAC (n = 23)_ | 45% to 53% | 50% to 54% | 38% to 52% | |
| 3) RFX (n = 21) | 79% to 90% | 82% to 90% | 78% to 85% | 0.035 |
| | | | | (RFX |
| | | | | to LAC) |

### INCORPORATION BY REFERENCE

The contents of all references, patents, pending patent applications and published patents, cited throughout this application are hereby expressly incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of improving the survival outcome for a subject suffering from hepatic encephalopathy (HE), comprising administering a composition comprising rifaximin to the subject.

2. The method of claim 1, wherein the survival outcome is improved by at least about 5%, 10%, 25%, 50% or 75% relative to a subject that has not been treated with rifaximin.

3. A method of reducing the frequency of hospitalization visits by a subject suffering from HE, comprising administering a composition comprising rifaximin to the subject.

4. The method of claim 3, wherein the reduction in the frequency of hospitalization visits comprises a reduction in the frequency of hospital readmissions.

5. A method of maintaining normal daily caloric intake in a subject suffering from cirrhosis and hepatic encephalopathy, comprising administering a composition comprising rifaximin to the subject.

6. The method of claim 5, wherein the subject maintains at least about 75%, 80%, 85% or 90% of normal daily caloric intake.

7. The method of claim 5, wherein administration of rifaximin reduces the risk of developing sarcopenia in the subject.

8. The method of claim 5, wherein administration of rifaximin reduces the risk of suffering from malnutrition in the subject.

9. The method of claim 1, wherein the subject is not concurrently administered lactulose.

10. The method of claim 1, wherein the subject is administered rifaximin at a dose of about 50 mg to about 6000 mg per day.

11. The method of claim 1, wherein the subject is administered rifaximin at a dose of between about 100 mg and about 6000 mg; from between about 50 mg and about 2500 mg BID; from between about 50 mg and about 2000 mg TID; 200 mg TID; 200 mg BID or 200 mg QD.

12. The method of claim 1, wherein the subject is administered rifaximin at a dose of about 550 mg, 600 mg or 1650 mg TID, QD or BID.

13. The method of claim 1, wherein the subject is administered rifaximin at a dose of about 550 mg BID.

14. The method of claim 1, wherein the subject is administered the composition for between about 1 week and about 24 months.

15. The method of claim 1, wherein the subject is administered the composition for about 14 days.

16. The method of claim 1, wherein the subject is administered the composition for at least about six months.

17. The method of claim 1, wherein the subject is administered the composition for at least about twelve months.

18. The method of claim 1, wherein the subject is administered the composition for at least about 24 months.

19. The method of claim 1, wherein the subject is administered the composition for at least about 36 months.

20. The method of claim 1, wherein the subject is administered the composition for the duration of the subject's life.

21. The method of claim 1, wherein the composition does not comprise lactulose.
